(19) United States
(12) Patent Application Publication
(10) Pub. No.: US 2005/0278006 A1
(43) Pub. Date: Dec. 15, 2005

(54) Solar energy stored papers and their clinical applications

(76) Inventor: Yoshiaki Omura, New York, NY (US)

Correspondence Address: FROMMER LAWRENCE & HAUG, 745 FIFTH AVENUE- 10TH FL., NEW YORK, NY, 10151, US

(21) Appl. No.: 11/148,841

(22) Filed: Jun. 9, 2005

Related U.S. Application Data
(60) Provisional application No. 60/578,581, filed on Jun. 10, 2004.
(60) Provisional application No. 60/582,264, filed on Jun. 23, 2004.

(51) Int. Cl. A61F 7/00

(52) U.S. Cl. 607/96

(57) ABSTRACT

The invention refers to a method of treating medical conditions through the application of solar energy stored papers during sunrise or sunset comprising the steps of storing solar energy from a sunrise or sunset into a storage media and applying said storage media to an affected area of a patient to achieve a desired result.

### CROSS REFERENCE TO RELATED APPLICATIONS

A claim of priority is made to U.S. provisional applications Nos. 60/578,581 filed June 10, 2004 and 60/582,264, filed Jun. 23, 2004, the disclosures of which are incorporated herein by reference.

### FIELD OF THE INVENTION

The present invention is directed towards storing solar energy and using it in clinical applications.

### BACKGROUND OF THE INVENTION

During the late 1980s and early 1990s, while doing research on Qigong, the inventor succeeded in storing Qigong energy on various materials including papers such as 3 inch 5 inch index cards and band-aids (1-4). When the physiological effects of these papers were analyzed, it was discovered that two distinctively opposite types of Qigong energy exist. The one with highly beneficial effects is named Plus (+) Qigong Energy, which has the following characteristics (5-20):
1. Reduce or eliminate pain.
2. Improve circulation.
3. Increase muscle strength.
4. Increase drug uptake.
5. Etc.

The other, undesirable, completely opposite type of energy was named Minus (−) Qigong Energy. Minus (−) Qigong Energy is characterized by:
1. Increases or creates pain.
2. Creates circulatory disturbance.
3. Reduces muscle strength.
4. Inhibits drug uptake.
5. Etc.

The inventor's recent study on these 2 completely opposing Qigong energies is compared in the following tables:

TABLE 1
Comparison of Effects of the Application of
(+) or (−) Qigong Energy Stored Paper

| Beneficial Effects of (+) | Undesirable Effects of (−) |
|---|---|
| Qigong enery | Qigong energy |
| 1) Improve circulation | 1) Disturb or reduce circulation |
| (Vaso-dilation effect) | (Vaso-constriction effect) |
| 2) Reduce or eliminate pain | 2) Increase pain |
| 3) Increase muscle strength | 3) Decrease muscle strength |
| 4) Relax spastic muscles | 4) Increase muscle spasticity |
| 5) Enhance drug uptake | 5) Reduce drug uptake |
| 6) Reduce TXB1 in blood & | 6) Increase TXB2 in blood and |
| tissue | tissue |
| 7) Increase β-endorphin | 7) Decrease β-endorphin and |
| and kyotorphin in blood | kyotorphin in blood and tissue |
| and tissue | |
| 8) Often decrease homo- | 8) Often increase homocysteine |
| cysteine and homocystine | and homocystine levels in blood |
| levels in blood and tissue | and tissue |
| 9) Often decrease serum | 9) Often increases serum |
| triglyceride levels | triglyceride levels |
| 10) Often decrease | 10) Often increase the blood |
| abnormally increased blood | glucose levels in hyper- |
| glucose levels in hyper- | glycemic patients |
| glycemic patients | |
| 11) Often decrease systolic | 11) Often increase systolic |
| blood pressure in hyper- | blood pressure in hypertensive |
| tensive patients | patients |
| 12) Increase normal cell | 12) Decrease normal cell |
| telomere | telomere |
| 13) Reduce cancer cell | 13) Increase cancer cell |
| telomere | telomere |
| 14) Reduces 8-OH-dG, which | 14) Increases 8-OH-dG |
| increases in the presence | significantly, which indicates |
| of DNA mutation | an increase in DNA mutation |
| 15) Increases abnormally | 15) Reduced Folic Acid level |
| reduced Folic Acid level | below normal value |
| toward normal | |

Originally it was difficult to store the one surface completely with (+) Qigong Energy, but eventually, by the late 1980s, by detecting which part of the right and left hand emit the (+) energy and which part emits (−) Qigong Energy, it became possible to store one surface completely with (+) Qigong Energy. When one side of the paper such as a 3 inch×5 inch index card is stored only with (+) Qigong energy, the inventor found that the opposite side of the card always exhibited (−) Qigong Energy, and when the (+) Qigong Energy surface is applied to the patient all of the above-described beneficial effects can often be detected. Therefore, in treating pain or improving the circulation or selectively enhancing drug uptake to the pathological area, the simplest approach is to always ensure that the (+) Qigong Energy stored side is applied on the body surface above the pathological area to be treated.

Since the early 1990s, the Bi-Digital O-Ring Test has been successfully used in making quick noninvasive early diagnosis of cancer, cardiovascular disease, and Alzheimer's Disease, often before standard laboratory tests can detect pathology. Using the Bi-Digital O-Ring Test Resonance Phenomenon between 2 identical substances, normal and abnormal molecules can be identified noninvasively using the resonance phenomenon between two identical substances, i.e., between a control substance of known quantity and an identical substance that exists inside the body. The Bi-Digital O-Ring Test is also used to evaluate the degree of the potential effectiveness of (+) Qigong-Energy and the identification of potentially highly beneficial medications and optimal dosage for the individual patient, since these always make the Bi-Digital O-Ring Test strong positive (+). Any medication, food or anything directly contacting the body surface which is harmful to the body always makes the Bi-Digital O-ring Test a strong negative (−), and the degree of negativity (−) as well as the degree of positivity (+) can be graded using the Bi-Digital O-Ring Test to compare their potential effectiveness or harmfulness.

### SUMMARY OF THE INVENTION

Various phases of solar energy were evaluated for possible medical application, using the Bi-Digital O-ring Test as set forth in U.S. Pat. No. 5,188,107 the disclosure of which is incorporated herein by reference. A 2-4 minute interval of highly beneficial phase during sunrise and sunset which is comparable or is stronger than (+) Qigong Energy was detected. This energy was stored on 3 inch×5 inch index cards. The sun energy stored on the exposed surface had a Bi-Digital O-Ring Test extremely strong positive (+) response, and the opposite side of the index card which was not exposed to the sun showed an equally strong negative (−) response. When the Bi-Digital O-Ring Test strong positive side (+) was applied to the patient's skin above various intractable painful areas with circulatory disturbances, including gangrenous pain, muscle pain, joint pain, & migraine headache, most of the pain disappeared or was significantly reduced within between 10 seconds and 5 minutes, with accelerated wound healing compared with Qigong energy stored paper of the same exposure, which caused pain to disappear within between 1.5 minutes and 15 minutes. When this Special Solar Energy Stored Paper was applied either directly to the skin above cancer positive areas or the midline of the upper chest above the thymus gland representation area, or the occipital area above the medulla oblongata, various cancer related parameters returned to close to normal values, with immediate clinical improvement. The beneficial effects of 10-60 seconds of application of the Special Solar Energy Stored Paper lasted for between 7 and 40 days, depending on the individual and their environmental electromagnetic field, how the special solar energy was stored, and how it was applied to the patient.

### DETAILED DESCRIPTION OF THE INVENTION

While the inventor was on an airplane from New York to Detroit, he was sitting in the left window side of the plane during sunset. He observed the sunset color above the clouds and suddenly thought about the possibility of whether the strong reddish light of the sunset above the clouds as seen from the airplane might also have positive energy such as (+) Qigong Energy. The inventor examined the red light coming from the sunset and found that during a short period of time it had Bi-Digital O-Ring Test extremely strong positive (+) response. Therefore, he thought this solar energy might have a somewhat similar effect to (+) Qigong energy stored paper. In order to test this hypothesis, he looked for white paper without any printing. The only paper with minimum printing available was the small paper napkin provided by the airlines accompanying the soft drink. Therefore the inventor selected this Bi-Digital O-ring Test extremely strong positive (+) period of the direct sunlight of the sunset and exposed the paper napkin, and then kept this paper between the pages of a book.

The next day he tested to see if the solar stored energy had a similar characteristic to (+) Qigong energy stored paper, namely that while one side is positive (+), the opposite side of the paper is always (−) negative. The one side exposed to the reddish sunset light had very strong positive (+) energy like (+) Qigong energy stored paper, and the other side had very strong negative (−) polarity. Qigong energy stored paper is often very effective in reducing pain, reducing circulatory disturbances, enhancing muscle strength and drug uptake. Since he did not have any severe pain, the inventor applied the paper to the prostate cancer positive area. In addition to adenocarcinoma of the prostate gland, the inventor also had adenocarcinoma of the colon, and squamous cell carcinoma of the right lung, all of which had been successfully controlled for the past 10 years. Whenever the inventor forgot for more than a few days to take his medication of EPA with DHA and cilantro tablet with the selective drug uptake enhancement method, the parameters associated with cancer such as Integrin α5β1, Oncogene C-fos Ab2, Hg, Cancer Telomere, 8-OH-deoxy Guanosine (8-OH-dG) often increase, while Acetylcholine of cancer tissue is markedly reduced and normal cell Telomere is also reduced.

When the inventor was traveling he often neglected to take these safe natural medications combined with the selective drug uptake enhancement method, which was originally discovered by the Inventor in 1990 and later refined, to deliver the drug selectively to the pathological area. As a result of this neglect, abnormal parts of the body frequently develop abnormal tingling sensations and an annoying dull pain. When this happens all cancer-related markers are slightly or moderately increased. Therefore the inventor applied this Bi-Digital O-Ring Test-strong positive (+) Special Solar Energy Stored Paper on the area above the prostate cancer positive area. All of the cancer related parameters tested became practically zero not only above the prostate cancer positive area but also the relatively benign squamous cell carcinoma of the right upper lung and the adenocarcinoma of the colon positive area. All these cancer-associated abnormal markers almost instantaneously disappeared, even though the paper was not applied above the squamous cell carcinoma of the lung or adenocarcinoma of the colon. This effect lasted for 40 days.

Based on this finding the inventor explored a variety of methods of storing solar energy and he examined the effect of the application of the Bi-Digital O-Ring Test strong positive (+) Special Solar Energy Stored Paper on volunteer patients with intractable medical problems, including severe intractable pain (due to gangrene, migraine headache, joint pain, and severe circulatory disturbances), various cancers, and chronic intractable recurrent bacteria and viral infections after informed consent forms were signed by the patients and witnesses. Some of the effects of Special Solar Energy Stored Paper on pain, circulatory disturbances, cancer, and infection will be discussed below.

### MATERIALS & METHODS

Since the initial discovery of the unexpected powerful therapeutic effect of the Bi-Digital O-ring Test strong positive (+) portion of sunset light of solar energy seen above clouds the inventor did not have a chance to take another airplane for almost 2 months. He made a similar preparation of Bi-Digital O-Ring Test strong positive (+) Special Solar Energy Stored Papers from his apartment window. Bi-Digital O-Ring test strong positive (+) special solar energy as identified by the Bi-Digital O-Ring Test, while cosmic and other background radiation as measured by Geiger Counter was very low and constant, was stored on 3 inch×5 inch index cards exposed for about 2 minutes. At ground level, when the Geiger counter reading suddenly increases more than several counts, the solar energy often becomes negative. In order not to confuse the positive (+) and negative (−) sides, the side of the index cards with parallel lines was exposed to the strong beneficial positive (+) solar energy. For safekeeping, as we had been doing with Qigong energy stored paper, this Special Solar Energy Stored Paper was completely wrapped and rewrapped with aluminum foil, with no gap or opening. By completely wrapping with aluminum foil we have been able to go through X-ray screening devices in airports without any harm to the Special Solar Energy Stored Paper, as was the case for Qigong energy stored paper since the late 1990s. As with Qigong energy stored paper, if the Special Solar Energy Stored Paper is directly exposed to a strong electromagnetic field, including the light of a flash for taking pictures, the stored energy will be instantaneously dissipated, and, therefore, it is not desirable to take a picture while paper is applied for treatment.

Subsequently, the inventor had the chance to prepare a variety of solar energy stored papers under different conditions, which included strong direct sunlight captured and stored on an airplane enroute, strong direct sunlight captured and stored on the ground and the special periods of sunrise and sunset. It should be noted that most cases were treated with the Bi-Digital O-Ring Test strong positive (+) Special Solar Energy Stored Paper which was prepared with indirect sunlight during sunset at the inventor's apartment.

Clinical Case 1: Coexistence of Multiple Cancers: Adenocarcinoma of the Colon, Adenocarcinoma of the Prostate Gland, & Squamous Cell Carcinoma of the Lung

The first case was the inventor who was a 69-year old male physician with an early stage of adenocarcinoma of the prostate gland, a well-controlled early stage of adenocarcinoma of the colon at the ascending colon near the cecum for the previous 10 years, and a relatively benign squamous cell carcinoma of the right upper lung. He had previously observed at times an abnormal tingling sensation around the cancer positive areas, often accompanied by very annoying dull pain. His various cancer related marker readings were often moderately increased. Observing such a sensation, he first measured the cancer related parameters, then made a 1-minute application of the Bi-Digital O-Ring Test strong positive (+) side of the Special Solar Energy Stored Paper (prepared on the airplane during the Bi-Digital O-Ring Test strong positive (+) phase of sunset above the clouds) on the prostate cancer positive area on the right lower abdomen. After application of (+) side of spiral solar energy stored paper, he then made another series of measurements of the same cancer-related parameters. The cancer-related parameters, moderately elevated before the 1-minute application of the Bi-Digital O-Ring Test strong positive (+) side of the Special Solar Energy Stored Paper, were markedly shifted toward the normal almost immediately following application. When the paper was reexamined after application on the abnormal area, both surfaces of the paper had became strongly negative (−). The following cancer marker improvements were observed, as shown in the following table:

| | | After Application of | |
|---|---|---|---|
| Before Application of (+) Solar | | the (+) Solar Energy | |
| Energy Stored Paper on Cancer | | Stored Paper on Cancer | |
| Positive Area | | Positive Area | |
| 1. | Integrin α5β1: 320 ng→ | <<1 | yg |
| 2. | Oncogene C-fosAb2: 320 ng→ | <<1 | yg |
| 3. | Hg: 300 mg→ | <<1 | yg |
| 4. | Acetylcholine: 0.1 pg→ | 50 | μg |
| 5. | Virus Infection: ↑↑↑↑↑↑→ | 0 | |
| 6. | Telomere (TTAGG): I200 ng→ | <<1 | yg |
| 7. | Normal Cell Telomere (TTAGG): | 250 | ng |
| | 150 ng→• | | |
| 8. | 8-OH-deoxy Guanosine: 80 ng→ | 1 | pg |
| 9. | Folic Acid: 125 ag→ | 50 | ng |
| 10. | TXB₂: 1180 ng→ | 0.5 | ng |
| 11. | Bi-Digital O-Ring Test without any | 0 | |
| | reference control substances: −6→ | | |

Similar strong improvement was found not only on the prostate cancer positive area but also in the adenocarcinoma of the colon positive area and squamous cell carcinoma of the lung positive area, in spite of the fact that the inventor did not apply the paper on the adenocarcinoma of the colon positive area or the squamous cell carcinoma of the lung positive area. The inventor, therefore, stopped all other treatment and examined these parameters daily to find out how long this highly beneficial effect would last. This highly beneficial response remained without any additional treatment for approximately 40 days, and then went back to the original abnormal condition.

In cancer cells, Integrin α5β1, Oncogene C-for Ab2, Hg, Telomere, and 8-OH-deoxy Guanosine are increased significantly with marked increase in viral infection, while Acetylcholine is markedly reduced and normal cell Telomere is moderately reduced. After application of the Special Solar Energy Stored Paper, all levels of abnormal molecules that appeared in cancer were reduced to much less than 1yg (where 1yg=10g⁻²⁴, which means practically no abnormal molecules exist). Acetylcholine levels, which in normal cells usually measure—1500 μg or higher, in any cancer cells are always reduced to an extremely low level of 1pg (1pg=10⁻⁹g) to 1ag (1 ag=I0⁻¹⁵g), in this case 0.1pg, but after application of the Special Solar Energy Stored Paper, it increased to 50 μg.

In the cancer tissue, cancer cell Telomere is always significantly increased to somewhere between 1000-1500ng. In this case, before application of the (+) Solar Energy Stored Paper the cancer cell Telomere was 1200ng, but after application of the Special Solar Energy Stored Paper, cancer cell Telomere was reduced to much less than 1yg (1yg=10⁻²⁴g), which means cancer cells can no longer divide, since living human cells require an average minimum of 100ng for the cell to be able to divide. Nornmal Cell Telomere, if it is reduced to about or less than 100ng means, on average, that most people will die within one year if nothing is done. As is clear from the above table, 150ng normal cell Telomere before application of the Special Solar Energy Stored Paper increased to 250ng after application of the paper. This 100ng increase is the equivalent of an average of about 10 years prolongation of life span.

8-OH-dG, which increases in the presence of DNA mutation, if it is increased over 80ng almost always indicates the existence of malignant tumor or serious cardiovascular disease, or excessive intake of Folic Acid. In this case Folic Acid in the cancer was extremely reduced to 10ag, which is very common in all cancer tissues, but after application of Special Solar Energy Stored Paper it increased to 50ng, which is within the normal range of 40-50ng.

Thromboxane B₂ (TXB₂) always increases in the presence of circulatory disturbances, and is the most reliable marker for detecting localized circulatory disturbances. In normal tissue TXB₂ is usually below 1-2ng, and TXB₂ at the normal parts of the inventor's body was an average of 0.5ng. At the prostate cancer positive area, however, before application of the Special Solar Energy Stored Paper, it was 1180ng, which indicates presence of severe circulatory disturbance. After application of the paper on the cancer positive areas, this value declined to a normal value of 0.5ng, indicating the severe circulatory disturbance at the cancer tissue was changed to normal circulation, accompanied by reduction of many cancer-related parameters toward normal.

These effects lasted 40 days. On the 41st day the abnormal tingling sensation accompanied by annoying dull pain reappeared at the right lower abdomen, particularly the right inguinal area. At that point more detailed measurement was performed at 4 different locations of the lower abdomen, as can be seen in the diagram accompanying the following table. As can be seen, the major abnormal sensation was felt in the prostate cancer positive 4 areas. The first measurement, taken 41 days previously, was performed only at Right PC 1, where maximum abnormal findings existed.

Prostate Cancer (PC): Various Cancer-Related Parameters Measured
Before & After Application of Special (+) Solar Energy Stored Paper

| | | Before Application of the Solar Energy | | | | | | | | After Application | |
|---|---|---|---|---|---|---|---|---|---|---|---|
| | | Stored Paper for 30 Seconds on PC1 & PC2 | | | | | | | | of the Paper | |
| | | Left PC1 | | Right PC1 | | Right PC2 | | Right PC3 | | All 4 Areas | |
| 1. | Integrin α5β1 | 260 | ng | 260 | ng | 250 | ng | 160 | ng | <<1 | yg (= 10⁻²⁴ g) |
| | (Normal: <1 ng) | | | | | | | | | | |
| 2. | Oncogene C-fos Ab2 | 260 | ng | 260 | ng | 265 | ng | 270 | ng | <<1 | yg |
| | (Normal: <1 ng) | | | | | | | | | | |
| 3. | Hg | 325 | mg | 360 | mg | 320 | mg | 310 | mg | <<1 | yg |
| | (Normal: <<1 mg) | | | | | | | | | | |
| 4. | Acetylcholine | 225 | fg | 50 | fg | 50 | fg | 50 | fg | 35 | μg |
| | (Normal: 1500 μg or +) | | | | | | | | | | |
| 5. | Telomere (TTAGGG) | 1400 | ng | 1400 | ng | 1300 | ng | 1200 | ng | <<1 | yg |
| | (Cancer Telomere) | | | | | | | | | | |
| 6. | PSA | 240 | ng | 470 | ng | 450 | ng | 440 | ng | 55 | pg |
| | (Normal: <100 pg) | | | | | | | | | | |
| 7. | TXB₂ | 1050 | ng | 1350 | ng | 1350 | ng | 1350 | ng | 0.5 | ng |
| | (Normal: <1-2 ng) | | | | | | | | | | |
| 8. | 8-OH-dG | 82 | ng | 82 | ng | 82 | ng | 82 | ng | 50 | pg |
| | (Normal: <3-5 ng) | | | | | | | | | | |
| 9. | Folie Acid | 110 | ag | 110 | ag | 110 | ag | 110 | ag | 50 | ng |
| | (Normal: 40-50 ng) | | | | | | | | | | |
| 10. | Vitamin B-12 | 20 | zg | 20 | zg | 20 | zg | 20 | zg | 1.5 | ng |
| | (Normal: 1-2 ng) | | | | | | | | | | |
| 11. | D, L-Homocysteine | 5 | mg | 5 | mg | 5 | mg | 5 | mg | 1.5 | ng |
| | (Normal: <0.5 mg) | | | | | | | | | | |
| 12. | Bi-Digital O-Ring | −6 | | −6 | | −6 | | −6 | | 0 | |

| Test Without Any Reference Control Substance (Normal: 0) | 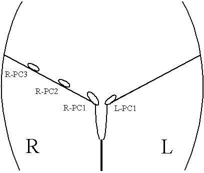 |
|---|---|
| *Telomere of the body is highest at birth. As age advances, and with rapid body growth, Telomere rapidly diminishes. After about 25.years of age the slope of age-dependent growth diminishes. In the 50s, Telomere will average ˜300-350 ng. In the 60s the average is ˜200-300 ng. Telomere declines further as age advances further. When Telomere | |
| # reaches 100 ng, most individuals will die within one year. Cancer Telomere is usually much higher (1000-1500 ng) than the normal body's Telomere. Among normal tissues, brain, heart, testes and ovaries are usually higher than the rest of the body. | |

At this time, 41 days after the initial application of Special Solar Energy Stored Paper, after all the beneficial effects disappeared, all four abnormal areas shown in the previous table were examined. Since at this time the inventor did not have the extra powerful solar energy stored paper made from direct sunset above the clouds from the airplane, he used the Special Solar Energy Stored Paper made from his high up apartment window during a sunset. The 3 inch×5 inch index card Special Solar Energy Stored Paper was applied for 30 seconds to cover both the right PC1 and right PC2. Within 1 minute after the 30-second application, all abnormal sensation disappeared. When the paper was removed, both sides of the paper had become Bi-Digital O-Ring Test strong negative (−). As can be seen in the preceding table, each parameter measured for the 4 different locations changed to the same improved value. For example, Integrin α5β1, Oncogene C-fos Ab2, Hg, and cancer cell Telomere all became much less than 1yg (1yg=10⁻²⁴g), Acetylcholine increased from initial 225fg measurement made at Left PC1 and 50fg measured at Right PC1, PC2, and PC3, to identical values of 35 μg. PSA, with 4 different values for each of the 4 locations, became an identical 50 μg. Thromboxane B2 was initially extremely abnormally high, with measurement between 1050ng to 1350ng, which means there was a severe circulatory disturbance at the cancer positive area, and all of these values declined to an identical 0.5ng, which is within the normal range of less than I-2ng. 8-OH-deoxy Guanosine was initially an identical 82ng at the prostate cancer positive 4 locations, but all 4 areas went down to normal value of 50 pg. In all four areas, Folic Acid was initially extremely low, 100ag, and after application of the paper all of these measurements increased to 50ng, which is within the normal range of 40-50ng. Vitamin B-12 was, at all 4 areas, initially the same 20zg, but after application of the paper, all became the same 1.5ng, which is within normal values. D, L-Homocysteine was initially 5 mg, which is very high, compared with a normal range of less than 0.5 mg, but it declined to 1.5ng. The Bi-Digital O-Ring Test without any reference control substance was (−)6, which is maximum abnormality, but all of these measurements at 4 locations reduced to 0, which is normal, with the application of the paper. These beneficial effects remained constant for 7 days without any significant changes, and on the 8th day it began to return to its previous abnormal state. Therefore, in comparing the effective duration between the 2 different solar energy stored papers under different circumstances, obviously the solar energy stored from the airplane during sunset seems to have a much stronger, longer-lasting effect than the one prepared from the inventor's apartment window through indirect sunset

Clinical Case 2: Early Stage of Breast Cancer, Intractable Gangrenous Throbbing Pain of Right 5th Finger, Pain of Ankle and Knee 33-year old Hispanic white female housewife (Maria F.) presented with non-healing ulcerative gangrene of reddish purple color in distal segment of right 5th finger, finger adjacent to the fingernail swollen to about 1.5 times normal diameter, all her fingers gnarled and swollen, with excruciating throbbing pain, the slightest touch causing extreme pain, with pain grading 9-10. Severe pain also in knees and ankles, the painful area with distinctive reddish discoloration, with a pain grading of from 9-10. Because of the pain in ankles and knees she had difficulty standing or walking. The pain in the right 5th finger was so severe that nothing could be touched. She had suffered from these problems for the past several years, with previous diagnosis by other physicians as mixed collagen disease with Raynaud's Disease and rheumatoid arthritis. In spite of the fact that she was getting a large amount of steroid hormones, 50 mg/day, the pain was not reduced. In addition, her face and body were pale white, as if she were anemic and suffered from malnutrition. The inventor had originally intended application of the positive (+) side of Special Strong Solar Energy Stored Paper (made from his apartment window for approximately 2 minutes exposure to a 3 inch×5 inch index card) on the painful 5^{th} finger for a period of 1 minute. However, within a few seconds of application she suddenly shouted “The pain is completely gone!” and dropped the paper. Both surfaces of the paper which she dropped on the floor now measured strongly negative (−). It was noted that the previous negative (−) Bi-Digital 0-ring Test abnormal findings were changed to 0, which usually means normal. All measurements are presented in the following table:

Bi-Digital O-Ring Test Grading Of Different Parts of the Body Before
& After Application of Special Solar Energy Stored Paper

| | | |
|---|---|---|
| | Immediately | 24 Hours |
| Before Application of | Following | After One |
| (+) Solar Energy | Application | Application |
| Stored Paper | of the Paper | of the Paper |

| | | | |
|---|---|---|---|
| Front of Body: | | | |
| R-Hand | −6 | 0 | 0 |
| L-Hand | −3 | 0 | 0 |
| Neck | −1 | 0 | 0 |
| Between Breasts | −6 | 0 | 0 |
| Umbilicus | −3 | 0 | 0 |
| R-Inguihal | −3 | 0 | 0 |
| L-Inguinal | −3 | 0 | 0 |
| R-Ear | −4 | 0 | 0 |
| L-Ear | −3 | 0 | 0 |
| Back of Body: | | | |
| R-Hand | −6 | 0 | 0 |
| L-Hand | −1 | 0 | 0 |
| Neck | −1 | 0 | 0 |
| Mid Back | −3 | 0 | 0 |
| Waist | −2 | 0 | 0 |
| Anus | −3 | 0 | 0 |
| R-Inguinal | −3 | 0 | 0 |
| L-Inguinal | −4 | 0 | 0 |

| | | Before | | After | |
|---|---|---|---|---|---|
| Normal Telomere | Right arm: | 110 | ng→ | 170 | ng |
| | Left arm: | 105 | ng→• | 170 | ng |
| 8-OH-deoxy Guanosine | Right arm: | 70 | ng→ | <1.0 | pg |
| | Left arm: | 70 | ng→ | <1.0 | pg |
| Folic Acid | Right arm: | 5 | ng→ | 50 | ng |
| | Left arm: | <0.5 | ng→ | 50 | ng |
| | Left leg: | 0.5 | ng→ | 50 | ng |

(0, −1, −2 = Within Normal Limits;
−3 = Borderline;
−4 = Mild Abnormality;
−5 Moderate Abnormality;
−6 = Strong Abnormality.
The Criteria For Cancer Screening With Integrin α5β1 60 ng)

A few weeks earlier, our non-invasive 2-minute cancer screening had indicated Integrin α5β1 was −6, which indicated a cancer-positive area existed somewhere in the right chest. Therefore the inventor used X-,Y-Axis Laser-Line Scanning of the upper part of the body with 60ng of Integrin α5β1 as reference control substance, and detected the exact location of the cancer positive area, which had an ellipsoidal shape, with a long axis of about 2 cm and a short axis of about 1 cm, but of slight thickness, at the right upper side of the breast near the axilla. A blood test was also performed for CA15-3 (Cancer Antigen 15-3), which is often increased if the breast cancer is well established, with significant mass. The inventor suggested that since it was an early stage, with a small mass, the blood test might not show a significant increase but there might be an increase slightly above the normal limit of the range. This laboratory test was performed 3 days before the stored Special Solar Energy Stored Paper application. As predicted, CA15-3 was 33.1 U/mL, which is slightly above the maximum normal range, compared with a normal range of 0.0-31.3 U/mL. At the same time CA125 (Cancer Antigen 125), which is often increased in the presence of ovarian cancer or uterus cancer was also measured, but it was within the normal limit at 31 U/mL compared with a normal range of 0.0-35.0 U/mL. Another significant blood test abnormality was that gammaglobin was extemely high, 3.5 g/dL compared with a normal range of 0.5-1.6 g/dL. Scrum albumen was markedly reduced to 3.2 g/dL compared with a normal range of 3.5-5.5 g/dL An additional significant abnormality was that AST(SGOT) was abnormally increased to 115 IU/L compared with a normal range of 0-40 IU/L, and ALT(SGPT) was also moderately increased to 68 IU/L compared with a normal range of 0-40 IU/L, and Creative Kinase (Total Serum) was a very high 925 U/L compared with a normal range of 24-173 U/L. Hemoglobin was reduced to 10.5 g/dL compared with a normal range of 11.5-15.0 g/dL, and hematocrit was also reduced to 32.0% compared with a normal range of 34.0-46.0%. An additional abnormality was that the Sedimentation Rate (by Westergren Method) markedly increased to 132 mm/hr compared with a normal range of 0-20 mm/hr. However, in spite of all these abnormalities, C-Reactive Protein was within normal limits at 3.7 mg/L, while the normal range is 0.0 4.9 mg/L. This low C-Reactive Protein may be due to abnormal liver dysfunction, as shown by abnormally increased AST and ALT, because C-Reactive Protein is produced by liver cells. This despite the claim by some of the leading cardiologists at Harvard and their supporters that C-Reactive Protein is the most reliable cardiovascular risk factor, as well as for inflammation, without simultaneously measuring and comparing L-Homocystine, the most reliable cardiovascular risk factor, which also reflects the presence of inflammation. Our previous study indicated that L-Homcystcine or L-Homocystinc are the more reliable cardiovascular risk factors as well as risk factors for inflammations. After application of positive (+) Special Solar Energy Stored Paper (obtained during strong Bi-Digital O-Ring Test positive phase of the sunset during very low cosmic background radiation as measured by Geiger Counter from the inventor's apartment window) to the area with severe throbbing pain of the 5th finger of the right hand with ulcerative gangrene, all the abnormal cancer markers of the right side of the breast almost instantaneously returned to close to normal values in spite of the fact that the paper was not applied to the right breast cancer positive area In addition, the non-healing ulcerating gangrenous wound was completely healed within 3 days. These relationships are shown in the following table:

Measurement of Cancer Parameters at the Breast Cancer Positive Area in the Side of the Right Breast Before & After Application of Special (+) Solar Energy Stored Paper

| | Before | | After | | 1 Day | |
|---|---|---|---|---|---|---|
| | Application | | Application | | Following | |
| Integrin α5β1 | 250 | ng | <<1 | yg | <<1 | yg |
| Oncogene C-fos Ab2 | 250 | ng | <<1 | yg | <<1 | yg |
| Hg | 300 | mg | <<1 | yg | <<1 | yg |
| Acetylcholine | 0.1 | pg | 40 | μg | 50 | μg |
| Telomere | 1300 | ng | <<1 | yg | <<1 | yg |
| Normal Tissue Telomere | 105 | ng | 200 | ng | 250 | ng |
| 8-OH-deoxy Guanosine | 110 | ng | 3 | ng | 3 | ng |
| Folic Acid | 1 | fg | 45 | ng | 45 | ng |

Clinical Case 3: Juvenile Alzheimer's Disease of Young College Student

This 21 year old Oriental female student majoring in Electrical Engineering at a major university had been aware of extreme fatigue for more than 1 year. For the preceding 4 months she had had difficulty in concentrating and memorizing new things. She was examined at the Stanford University Clinic by a number of physicians, but all laboratory tests, including blood tests and brain MRI, showed no abnormality. A professor from NYU brought her for evaluation as to the possible cause of her problem and, hopefully, for an approach to treatment. Bi-Digital O-Ring Test evaluation of the hippocampus area indicated Acetylcholine was at an extremely reduced level of 5 μg.

| | | | | 24 Hours | | After Stored | |
|---|---|---|---|---|---|---|---|
| Pathological Factors | | Initial | | After | | Solar Energy | |
| At R- & L-Hippocampus | | Evaluation | | Treatment | | Application | |
| Acetylcholine: | R- & L-Sides: | 5 | μg | 10 | μg | 800 | μg |
| (β-Amyloid | R-Side: | 7 | ng | 1 | ng | <0.1 | ng |
| (1-42): | | | | | | | |
| | L-Side: | 8 | ng | 1 | ng | <0.1 | ng |
| Al: | R- & L-Sides: | 250 | mg | 1 | mg | <<1 | yg |
| Hg: | R- & L-Sides: | 250 | mg | 1 | mg | <<1 | yg |
| ThromboxaneB2(TXB2): | | 550 | ng | <1 | ng | <<1 | yg |
| Chlamydia Trachomatis: | | 1700 | ng | <1 | ng | <<1 | yg |
| Mycobacterium | R-Side: | 30 | μg | 1 | ng | <<1 | yg |
| Tuberculosis: | L-Side: | 49 | μg | 1 | ng | <<1 | yg |

| Amoxicillin Sensitive | ++++++ | ++ | 0 | |
|---|---|---|---|---|
| Bacterial Infection: | | | | |
| EPA/DHA Sensitive | ++++++ | ++ | 0 | |
| Viral Infection: | | | | |
| Bi-Digital O-Ring Test: | −6 | −3 | 0 | (+3) |
| (Without Any Substance) | | | | |

Acetylcholine in the forehead was slightly better than that in the hippocampus, but still a very low 50 μg, compared with a normal value for Acetylcholine in any part of the brain of 1500 μg or higher. Brain dysfunction appears when Acetylcholine is less than 500 μg. When Acetylcholine is less than 200-300 μg, most observers recognize a problem in brain function. In Alzheimer's Disease the inventor's previous study indicated that when insoluble β-Amyloid (1-42) is over 4-5 ng most people show some type of memory deficiency. If this happens in the classroom, for example, students often ask the same question again and again. Normal β-Amyloid (1-42) level is less than 1-2 ng. In Alzheimer's Disease, β-Amyloid (1-42) is always increased over 7ng. In my previous study using the Bi-Digital O-Ring Test Resonance Phenomenon Between 2 Identical Substances to detect the amount of various substances, in Alzheimer's Disease cases Al is always higher than 250 mg, β-Amyloid (1-42) is 7ng or higher, and Acetylcholine is less than 100 μg. Until only a few years ago, no one knew the cause of the increased β-Amyloid (1-42) levels observed. The inventor's study, however, demonstrated that all the Alzheimer's patients he examined had mixed bacterial viral infections. When these severe mixed infections were treated and nearly completely eliminated, β-Amyloid (1-42) became normal, with corresponding improvement of memory function. Further, when more than 80-90% of metal deposits of Al and Hg are removed, Acetylcholine increases by 2-3 times. In Alzheimer's patients the inventor had found that when Chlamydia Trachomatis infection and Mycobacterium Tuberculosis infection were eliminated, β-Amyloid (1-42) markedly diminishes, to within the normal range. Thromboxane B2 is normally less than 1 ng, and ideally should be 0. However, when there is a circulatory disturbance, it is markedly increased. When a different part of the brain in this patient was evaluated, Thromboxane B2 was on average 550ng, which indicated the existence of very significant circulatory disturbance throughout the brain.

Once the information listed in the table above was determined, as a first step of treatment, the inventor decided to eliminate excessive metals, since in the presence of excessive metal no antibiotics or antiviral agents can work efficiently. Therefore, the inventor first gave Cilantro to remove excessive levels of Al and Hg. The inventor elected to give her, shortly after first examination of the patient in late afternoon the following 3 compatible medications including: 1) Cilantro tablet, 100 mg; 2) Mixture of EPA, 180 mg with DHA, 120 mg; 3) Trimox, 500 mg, with the Selective Drug Uptake Enhancement Method to selectively deliver medication to the pathological area while drastically reducing the drug uptake to normal body tissues. The Selective Drug Uptake Enhancement Method was first discovered by the inventor in 1990, and the method has been continuously refined until the present time, using either mechanical, electrical, electromagnetic field (including red light from light emitting diode or soft laser), or thermal stimulation. Stimulation was persistently applied to the entire brain representation areas at the distal segment of right and left middle fingers. Treatment was repeated in the course of the evening, employing the same complementary medications, both to preclude any possible problematic drug interactions, and to take advantage of the synergistic effects of this particular combination. Treatment, with continued stimulation of the brain representation areas over a time-span of several hours, was again repeated around midnight, before the patient went to bed. Generally, with one or two administrations of Cilantro employing the Selective Drug Uptake Enhancement Method, more than 80-95% of the metal will have been removed by the following morning. It was necessary to discontinue continued administration of the Cilantro the following morning in this case, because Cilantro is not compatible with the medications most frequently used to treat Chlamydia Trachomatis, and Mycobacterium Tuberculosis, like Isoniazid and Rifampin, (which are also incompatible with many other antiviral and antibacterial agents). It is, for this reason, generally impossible to simultaneously treat viral and bacterial infections, since, on account of the drug interaction, both effects will be cancelled.

To address this problem the inventor in the mid- 1990s evaluated by using the Bi-Digital O-Ring Test about 150 Japanese herb medicines, known as Kampo Medicine, a Japanese form of traditional Chinese herb medicine, manufactured by Tsumura Pharmaceutical Company in Japan. At that time the inventor found Saiko-Keishi-To, among these herb medicines, also listed and referred to as Number 10. to be a highly effective as anti-tubercular medicine. It is also compatible with antiviral agents, including EPA and DHA, as well as the antibacterial agent Trimox, and Substance Z, the principal component of which is extracted from the Indigo plant, and which the inventor discovered in collaboration with Hayashibara Biochemical Company, and which is very effective for treatment of Chlamydia Trachomatis (about 750 times more effective than Azithromycin, which is considered to be one of the best available antibiotics for Chlamydia Trachomatis). Therefore, the patient started taking, in the morning, EPA and DHA as antiviral agents, Trimox and Substance Z for Chlamydia Trachomatis, and Number 10 for Mycobacterium Tuberculosis. She continued this treatment until late afternoon, and, about 24 hours after the initial evaluation, the inventor re-evaluated the changes, indicated in the previous table.

As a result of the application of the Selective Drug Uptake Enhancement Method together with effective medications, improvement was very significant within only 24 hours. Of vital importance, however, Acetylcholine in the hippocampus area had only increased to 10 μg, which is the lower end of the range typical of Alzheimer's patients, who usually have less than 100 μg. It is necessary to raise this level to more than 500 μg in order to realistically expect significant improvement in memory, despite the fact that β-Amyloid (1-42) had entered the normal range, and Al and Hg had become reasonably low. At this point, therefore, the inventor decided to apply Special Solar Energy Stored Paper on the patient's occipital area, above the Medulla Oblongata, where the circulatory center of the Medulla Oblongata is located. As soon as the paper had been applied for 10 seconds there was a sudden, dramatic improvement in circulation. Thromboxane B2 declined to less than 1ng from 550ng, while Acetylcholine at the hippocampus increased from 10 μg to 800 μg. Acetylcholine at the forehead, originally 50 μg, which had been at that point the highest level in the entire brain, had increased to 100 kg after 24 hours of medication via the Selective Drug Uptake Enhancement Method. However, immediately after application of the Special Solar Energy Stored Paper above the Medulla Oblongata area, Acetylcholine increased to 800tg level at most of the brain. After 24 hours of treatment via the Selective Drug Uptake Enhancement Method, her abnormal sensation of fatigue had vanished, and the quality of her sleep improved. Prior to treatment she suffered from insornnia, awakening frequently in the middle of the night. After the 10-second application of the Special Solar Energy Stored Paper, all abnormal components of Alzheimer's Disease were completely eliminated. Her circulation markedly increased, and Acetylcholine now at 800 μg, she found it easy to concentrate, and fatigue was significantly reduced.

Twenty-four hours earlier, she was very quiet and rarely spoke with others except for the minimum conversation necessary for the inventor's questions. After 10-second application of the Special Solar Energy Stored Paper, her pale white face became more pinkish, and she became more talkative and cheerful. She could easily concentrate, and could memorize and think more effectively. Three days later, Acetylcholine of the brain averaged 850 μg, which indicates further slight improvement in the amount of Acetylcholine. The patient indicated that she can function much better, and can concentrate and memorize much more effectively. While before the initial examination, and 24 hours after first treatment, she did not ask any questions concerning her condition, 3 days after 10-second application of the Special Solar Energy Stored Paper she began to actively ask many questions about her condition, and explained very clearly what improvement she had noticed.

Clinical Case 4: Hypertension & Occipital Headache

This 24-year old white male engineering school graduate has had high blood pressure of 160-110 mm Hg for the past year. Originally, he was prescribed a number of anti-hypertensive drugs but they did not lower his blood pressure. As a result he was not taking any medication. His headache was located in the left occipital area, and he had pain in the left shoulder, left upper back, left upper chest, and the entire arm, particularly on the palm and palm-side of the arm. This painful area had Herpes Simplex Virus Type II: 250ng, and Chlamydia Trachomatis: 900ng, and Mycobacterium Tuberculosis of 25 μg was found in this painful area. Since this patient came very late, a few hours after the conference was finished, I saw him about 9 pm and instead of initiating our regular treatment, we gave him Special Solar Energy Stored Paper on the skin above the Medulla Oblongata for a few seconds. That eliminated most of the left occipital headache, but he was still having pain in the left shoulder and left upper chest and arm. Another piece of the Special Stored Solar Energy Paper was applied to the left neck and shoulder area, which reduced the blood pressure from the original 160-110 mm to 125-75 mm Hg, and 80% of the pain disappeared. The next day blood pressure remained normal, and most of the occipital headache was gone, and about 80% of the pain in neck, shoulder, and arm also disappeared, and all of the abnormal micro-organisms we found before application of the Special Solar Energy Stored Paper could no longer be detected. In order to prevent possible return of the infections the inventor asked the patient to take a capsule of EPA 180 mg with DHA 120 mg as safe and effective antiviral agents, and Substance Z as safe and effective anti-Chlamydia agent 3 times a day, and Saiko-Keishi-To as a safe and effective anti-Tuberculous medication 2 times a day with Selective Drug Uptake Enhancement Method, since the inventor would not see the patient for more than one month. One month later the improvement remained without headache and hypertension.

Shortly after this case, the inventor treated an approximately 75-year old housewife with a history of more than 10 years of essential hypertension. Both systolic and diastolic blood pressure were similar to this patient. The inventor found multiple bacterial and viral infections in this patient at the entire Medulla Oblongata. When the inventor applied the Special Solar Energy Stored Paper on this patient, all infections disappeared immediately, but no improvement in blood pressure was observed. The pre-treatment pathological finding was characterized by Herpes Simplex Type 2 virus with 700 ng infection, Chlamydia Trachomatis 500 μg, and Mycobacterium Tubercolosis 5-20 μg all of which can increase blood pressure and can create pain on the occipital area, shoulder and arm. The Medulla Oblongata which has the cardiovascular control center with the left half thereof being infected. In early 1990's the inventor found that many patients with intractable hypertension which appeared following occipital headache was often due to mixed bacterial and viral infection of Medulla Oblongata.

Other cases of treating various ailments are documented in the publication Acupuncture & Electro-Therapeutics Research, Vol. 29 at pp. 1-42, the disclosure of which is incorporated herein by reference. Thus by the present invention its objects and advantages will be realized. Modifications thereof will be apparent to those skilled in the art.

1. A method of treating medical conditions through the application of solar energy generated during sunrise or sunset comprising the steps of storing solar energy from a sunrise or sunset in a storage media; and applying said storage media to an affected area of a patient to achieve a beneficial result.

2. The method as claimed in claim 1 wherein the storage media is paper.
